(19) United States
(12) Patent Application Publication
(10) Pub. No.: US 2006/0281986 A1
(43) Pub. Date: Dec. 14, 2006

(54) Apparatus and methods useful for monitoring intraocular pressure

(75) Inventors: Werhner C. Orilla, Anaheim, CA (US); James A. Burke, Santa Ana, CA (US); Amol D. Kulkarni, Pune (IN)

Correspondence Address: Stephen Donovan;Allergan, Inc., 2525 Dupont Drive, Irvine, CA, 92612, US

(73) Assignee: ALLERGAN, INC., Irvine, CA (US)

(21) Appl. No.: 11/149,581

(22) Filed: Jun. 9, 2005

(51) Int. Cl. A61B 3/16

(52) U.S. Cl. 600/398

(57) ABSTRACT

Apparatus useful in sensing intraocular pressure are provided. The apparatus generally include a rigid tube defining a hollow through space sized and adapted to allow a flexible catheter of a pressure sensor or transducer used to sense IOP to pass in or in fluid communication with the hollow through space. The apparatus includes stabilizing structure for facilitating fixing of the tube in a desired position or angular orientation within the eye. The present apparatus more effectively maintains the position or angle of the flexible catheter or distal end portion of the pressure sensor or in the eye relative to a similar pressure sensor transducer including a flexible catheter without the rigid tube and/or without the stabilizing structure.

The present invention relates to apparatus and methods useful in measuring/monitoring intraocular pressure. More particularly, the invention relates to such apparatus and methods which are very useful in measuring and/or monitoring intraocular pressure in humans and animals, such as rabbits, primates and the like, over long periods of time.

Continuously measuring/monitoring intraocular pressure (IOP) in test animals is very desirable in various instances, for example, to monitor the course of a disease, such as glaucoma, and/or a treatment of such a disease. Such IOP measuring/monitoring often occurs over a long period of time, for example, over about 3 months to about 1 year or about 2 years or longer.

Such IOP measuring/monitoring is often conducted using a pressure sensor or transducer which is implanted into the body of the subject. This sensor/transducer transmits signals indicative of the IOP to a remote receiver, for example, to a receiver placed in a cage housing the animal subject, which receives and collects the IOP data for analysis. The implanted pressure sensor/transducer includes a flexible polymeric, e.g., silicone, catheter which is, for example, surgically inserted into the sclera of the subject's eye. This allows the sensor to sense pressure from within the eye of the subject. Prior art methods involve heat bending the catheter at a 90° angle, and then placing the distal end or tip of the catheter into the vitreous.

One problem with such techniques is that the angle of insertion of the catheter is not stable due to the flexibility of the catheter tubing. Especially with the movements of the eye, the angle of insertion of the catheter has a tendency to change, which can cause the catheter to touch either the lens or the retina of the eye. Such change and/or touching can compromise IOP data collection. For example, the sensor may lose its capability to collect correct eye pressure data when the tip of the catheter is occluded by tissue from the lens or retina of the eye.

In addition, pressure sensors/transducers commonly used to monitor IOP have relatively limited useful lives, for example, on the order of about six (6) months or less. Since it is often useful to monitor IOP for longer periods of time, this limitation of the current sensors/transducers represents a substantial problem.

It would be advantageous to provide pressure sensors/transducers with enhanced position stability in the eye and/or with longer useful lives.

### SUMMARY OF THE INVENTION

New apparatus and methods for use in sensing, measuring and/or monitoring intraocular pressure (IOP) have been discovered. The present apparatus effectively provide enhancement in performance and use relative to prior art devices. For example, the present apparatus are structured to more effectively maintain the position of the sensor tip in the eye to allow more reliable IOP data to be obtained over relatively long periods of time. In addition, the present apparatus preferably are structured to be useful for longer periods of time relative to prior art systems. The present methods employ apparatus in accordance with the present invention and provide substantial benefits. The present apparatus and methods are relatively easy and cost effective to produce and practice and are very effective in use.

In one broad aspect of the present invention, apparatus are provided for use in sensing, measuring and/or monitoring IOP. The apparatus comprises a rigid tube including a first portion and a second portion positioned at a fixed angle relative to the first portion. The tube defines a hollow through space sized and adapted to allow a flexible catheter of a pressure sensor or transducer used to sense IOP to pass in or in fluid communication with the hollow through space. The present angularly oriented rigid tube more effectively maintains the position or angle of the flexible catheter or distal end portion of the pressure sensor or transducer in the eye relative to a similar pressure sensor or transducer including a flexible catheter without the rigid tube. This is a substantial advantage and provides for more reliable IOP sensing, measuring and/or monitoring, particularly over relatively long periods of time, for example, on a substantially continuous basis.

In a useful embodiment, the rigid tube has a distal end configured to be inserted into an eye of a human or animal subject. The tube may have a distal end which is beveled, for example, to facilitate passing the distal end portion of the tube through an incision in the eye.

The fixed angle between the first and second portions of the rigid tube may vary over a wide range. What is important is that the angle be fixed to facilitate maintaining the distal end portion of the pressure sensor, for example, the distal end or tip of the catheter of the pressure sensor, at a location and/or an angle, for example, a substantially fixed location and/or angle, in the eye. The angle between the first and second portions of the rigid tube may be in the range of about 15° or about 30° or about 45° or about 60° or about 75° to about 165° or about 150° or about 135° or about 120° or about 105°. In one particularly useful embodiment, the angle between the first and second portions is about 90°.

In one embodiment, the tube is derived from a needle or portion thereof, for example, a needle sized and structured as a conventional G19 needle, a needle sized and structured similar thereto, other suitably sized and structured needles and the like. Although it may be useful to form the present rigid tube from a needle, the present invention is not limited to rigid tubes derived from needles.

The rigid tube may be made of any suitable material. Preferably, the tube is biocompatible, that is the tube is made of a material which does not substantially react or interfere with the body, for example, tissue, of the subject and/or is substantially not toxic to the body, for example, tissue, of the subject in which the tube is to be placed or which the tube contacts. Thus, in a preferred embodiment, the rigid tube comprises a biocompatible metal. In other embodiments, the tube comprises one or more glasses, advantageously biocompatible glasses, for example, borosilicate glasses, and the like and mixtures thereof.

In a useful embodiment, the apparatus further comprises an enlarged stabilizer, for example, an enlarged stabilizer member, secured to the tube. The stabilizer may be configured to be effective in maintaining a desired orientation, for example, angular orientation, or position of the tube in an eye into which the tube is inserted, introduced or placed. In some embodiments of the invention, the stabilizer member is structured to facilitate anchoring of, for example, suturing, the apparatus to sclera. Both the tube and the stabilizer may be, and preferably are sized and structured to be located within the body of the human or animal subject. The tube and stabilizer may comprise the same or different materials. In a very useful embodiment, the stabilizer comprises a biocompatible material, for example, a biocompatible polymeric material. In some embodiments of the invention, the stabilizer comprises one or more metals, advantageously biocompatible metals, such as surgical grade stainless steel, gold, and the like and mixtures thereof; and/or glasses, advantageously biocompatible glasses, for example, borosilicate glasses and the like and mixtures thereof.

The stabilizer may, and preferably does, substantially surround a region of the tube at which the first and second portions of the tube meet. In other embodiments, the stabilizer is adjacent a region of the tube wherein the first and second portions meet. Such placement of the stabilizer is very effective in maintaining the angular orientation or position of the distal end portion of the tube in the eye.

In some embodiments of the invention, the stabilizer is structured or shaped to have one or more substantially flat or planar surfaces. For example, the stabilizer may be disk shaped. In other embodiments of the invention, the stabilizer comprises a sheath circumscribing a portion of the tube, for example, a sheath having an outer surface having a shape substantially corresponding to the shape of the outer surface of the tube circumscribed by the sheath.

In another useful embodiment, the apparatus comprises a stabilizer portion and a tube portion depending therefrom. The stabilizer portion and the tube portion define a hollow through space sized and structured to allow a flexible catheter of a pressure sensor to pass in or in fluid communication with the hollow through space. In this embodiment, the tube portion is positioned at a given angle, for example, in a range of about 30° to about 150° or about 45° to about 135° or about 65° to about 105°, or about 70°, relative to the stabilizer portion in order to fix a distal end of the tube portion in a location away from a more sensitive part or parts, for example, a lens, of an eye, for example, while the apparatus is located in the eye.

In yet other embodiments of the invention, the apparatus comprises a rigid tube aligned along three different geometrical axes. For example, the rigid tube includes a first portion, a second portion and a third portion, each portion being disposed at an angle relative to each other portion. The third portion is located intermediate the first and second portions and functions as a stabilizer, for example, to stabilize the rigid tube in the desired angular orientation in the eye.

A flexible catheter of a pressure sensor used to sense IOP advantageously is pressure sealed relative to the rigid tube when the catheter is located in or in fluid communication with the hollow through space defined by the tube. The present apparatus preferably further comprises an adhesive component, for example, a biocompatible adhesive component positioned to pressure seal the flexible catheter relative to the tube when the catheter is located in or in fluid communication with the hollow through space formed by the tube.

In one very useful embodiment, the apparatus of the present invention further comprises a flexible catheter of a pressure sensor used to sense IOP. The catheter advantageously is biocompatible and is sized and structured to pass in, or in fluid communication with, the hollow through space of the tube. One or more additional components, or even all other components, of a pressure sensor used to sense IOP may be included within the present apparatus.

The pressure sensor advantageously is powered by a battery assembly. This is particularly advantageous when the entire apparatus is to be placed within the body of the subject.

As noted previously, the prior art pressure sensors or transducers have a substantial limitation in being useful for only a relatively short period of time, for example, for about six months or less. In the present apparatus, the pressure sensor is advantageously configured or structured to be powered by two (2) or more batteries in parallel with each other. It has been found that such a configuration allows the pressure sensor to remain operable for longer periods of time, for example, at least about six (6) months or about eight (8) months or about one (1) year or longer, relative to previous pressure sensors, for example, substantially identically structured pressure sensors, which included only a single battery.

This is an important feature of the present invention since relatively extensive surgery is required to place the apparatus in the eye of the subject. If the power source of the pressure sensor fails, the pressure sensor becomes ineffective and, if the period of time at which the failure occurs is not long enough, the IOP data itself may be useless. By providing an additional power supply as described herein, particularly in combination with a rigid tube and/or stabilizer as described herein, IOP data can be effectively and conveniently obtained over lengthy periods of time, as needed to meet the goals of the particular situation involved.

In one particularly useful embodiment, the entire apparatus, that is the rigid tube, stabilizer, pressure sensor and batteries, if any, are structured to be placed substantially completely in a body of a human or animal.

In one embodiment, the components of the pressure sensor other than the flexible catheter are located in a housing which is made of a biocompatible material, for example, a biocompatible metal, a biocompatible polymeric material and the like and combinations thereof.

In another broad aspect of the present invention, methods for sensing, measuring and/or monitoring IOP are provided. Such methods comprise placing a flexible catheter of a pressure sensor used to sense IOP in, or in fluid communication with, a hollow through space defined by a rigid tube including a first portion and a second portion positioned at a fixed angle relative to the first portion. The distal end portion of the rigid tube is introduced into an eye of a human or animal. The pressure sensor is employed to sense the IOP of the eye of the human or animal. In one very useful embodiment, apparatus in accordance with the present invention are employed in practicing the present methods. In one embodiment, the introducing or placing step is effective to locate the distal end portion of the tube into the vitreous of the eye.

Any and all features described herein and combinations of such features are included within the scope of the present invention provided that the features of any such combination are not mutually inconsistent.

These and other aspects of the present invention are set forth in the following detailed description and claims, particularly when considered in conjunction with the accompanying drawings in which like parts bear like reference numerals.

### BRIEF DESCRIPTION OF THE DRAWINGS

FIG. 1 is a side, somewhat schematic view of an apparatus for use in sensing intraocular pressure of the eye in accordance with the present invention.

FIG. 2 is a top view of a portion of the apparatus shown in FIG. 1.

FIGS. 3 and 4 are side views of the apparatus shown in FIG. 1 as it is being made.

FIG. 5 is a cross-sectional view of a mammalian eye having inserted therein a portion of the apparatus shown in FIG. 1.

FIG. 6 is a front view of the eye into which the apparatus shown in FIG. 1 is inserted.

FIG. 7 is a simplified diagram of an apparatus in accordance with the present invention which is structured to be substantially entirely located in a body of a human or animal.

FIG. 8 is a somewhat schematic view of a pressure sensor apparatus useful in the apparatus shown in FIGS. 1 and 7.

FIG. 9 is a side view of an alternate embodiment of the invention similar to the embodiment shown in FIG. 1.

FIG. 10 is a simplified diagram of another embodiment of the invention, similar to the embodiment shown in FIG. 9, showing the separate components of the embodiment.

FIG. 11 is a side view of a further embodiment of the invention including a sheath for facilitating anchoring of the apparatus in an eye.

FIG. 12 is a perspective view of an additional embodiment of the invention.

FIG. 13 is a perspective view of still another embodiment of the invention useful for sensing intraocular pressure in an eye.

### DETAILED DESCRIPTION

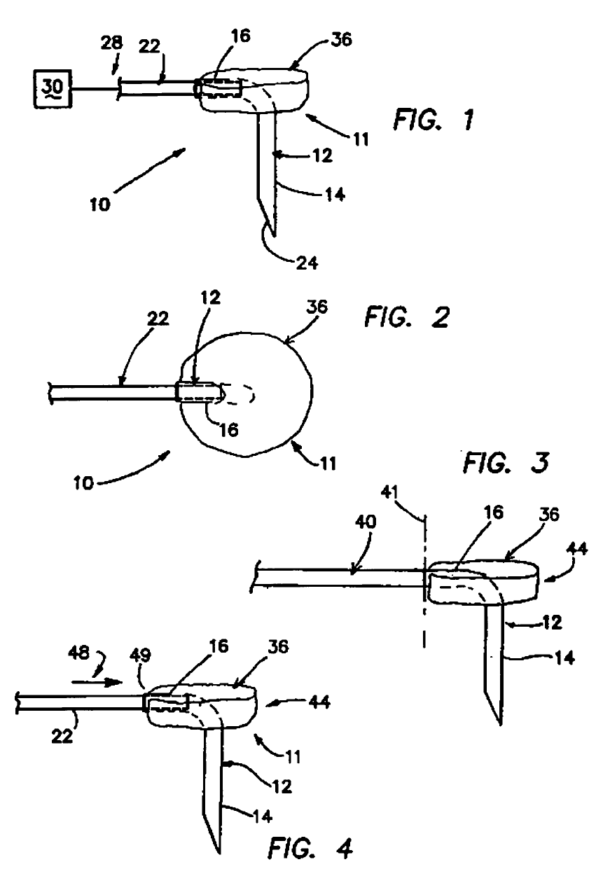

With reference to FIGS. 1 and 2, an apparatus in accordance with the present invention is shown generally at 10. The apparatus 10 generally includes a fixation assembly 11 structured to maintain the position and angular orientation of a pressure transducer catheter in an eye. In the embodiment shown, the assembly 11 comprises a rigid, hollow tube 12, for example, made of a biocompatible metal, including a first portion 14 and a second portion 16 positioned at a fixed angle relative to the first portion. In one embodiment, tube 12 is made from a G19 needle, for example, a G19 regular wall needle, and is gold plated. The tube 12 defines a hollow through space 17 sized and structured to allow a flexible catheter 22 to pass in or in fluid communication with the hollow through space. The tube 12 has a distal end 24 configured to be inserted into an eye of a human or animal. For example, the distal end 24 of the tube 12 may be beveled, as shown. In other embodiments of the invention, the distal tip is non-beveled, or blunt. The hollow through space 17 of the tube 12 may be sized to receive a conventionally sized catheter of a conventional pressure transducer used to sense/measure/monitor intraocular pressure (IOP).

The flexible catheter 22 may be a component of a pressure sensor assembly 28 including a pressure transducer 30 connected to the catheter 22 and effective in sensing/measuring/monitoring IOP when the distal tip of the catheter 22 is located in an eye, for example a human or animal eye.

In some embodiments of the invention, the pressure sensor assembly 28 is conventional in structure. For example, the pressure sensor assembly 28 may include a pressure radiotransmitter, for example, a PAC-40 pressure radiotransmitter manufactured by Data Science International, St. Paul, Minn. The pressure radiotransmitter contains a pressure transducer, an amplitude modulation radiotransmitter and a power supply in a biocompatible case or housing.

With particular reference to FIG. 2, the fixation assembly 11 of the apparatus 10 further includes a stabilizer member 36 secured to the tube 12 and effective in maintaining a desired position of the tube 12 in an eye into which the tube 12 is inserted. It can be appreciated that the stabilizer member 36 is enlarged in size compared to the relatively thin tube 12, for example, made of a G19 regular wall needle. The stabilizer member 36 preferably comprises a biocompatible material, preferably a biocompatible silicone polymeric material. Alternatively, stabilizer member 36 may comprise a biocompatible glass, for example, a borosilicate glass, a metal material, or other suitable biocompatible material. The stabilizer member 36 substantially surrounds a region of the tube 12 at which the first and second portions 14, 16 meet.

FIGS. 3 and 4 illustrate a suitable method of making apparatus 10. To form the tube 12, a G19 regular wall needle 40 is heated and bent to form first and second portions 14, 16, respectively. In the embodiment shown, the first and second portions 14, 16 are disposed at about a 90° angle relative to each other. The stabilizer member or element 36, for example, made of polymeric silastic material, is then placed on rigid tube 12, as shown. The needle 40 is then cut, for example, along line 41 closely adjacent the edge of stabilizer element 36, with a sharp implement in order to remove a proximal portion of the needle 40, as indicated in FIG. 3. After the needle is cut, the tube 12 comprises first portion 14 and second portion 16.

As shown in FIG. 4, the distal end of a flexible catheter, in this case, flexible silicone catheter 22, is inserted distally into the second portion 16 of the tube 12 as indicated by arrow 48. Preferably, the catheter 22 is pressure sealed relative to the tube 12 where the catheter 22 is located in the hollow space. For example, a biocompatible adhesive component 49 may be provided between an outer surface of the catheter 22 and an inner surface of the tube 12 to pressure seal the catheter to the tube and to secure the catheter to the tube. For example, the flexible catheter 22 may be pressure sealed in a fluid tight manner to the tube 12, for example, using a small amount of conventional biocompatible cyanoacrylate adhesive between the tube 12 and catheter 22.

Although the first portion 14 and second portion 16 of tube 12 are shown to be disposed at an angle of about 90° relative to each other, it is contemplated that the first and second portions 14, 16 may define other angles therebetween, for example, as described elsewhere herein. The selection of the angle between first and second portions, as well as an angle of the stabilizer member relative to the first and/or second portions, may vary depending upon the desired positioning of the apparatus 10 in the eye.

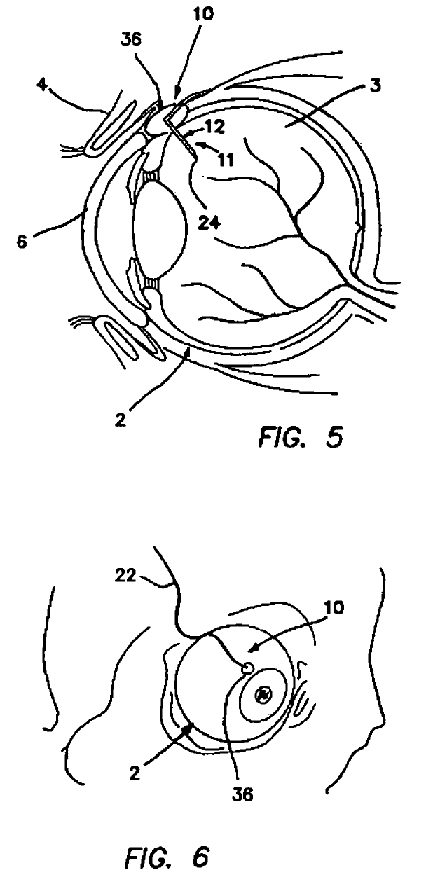

Some of the advantages of the present invention can be better understood with reference to FIGS. 5 and 6. As shown, apparatus 10, and in particular fixation assembly 11, is implanted in an eye 2 such that the distal end 24 of the tube 12 is located in the vitreous 3 of the eye. The apparatus 10 is implanted during a surgical procedure on the subject, e.g., rabbit or monkey. For example, the pressure transducer 30 is placed in a pocket formed in the underlying fascia of the scalp. The catheter 22 is then guided subcutaneously to the orbit. The distal end of the catheter 22 is then placed in and secured to the tube 12, as discussed elsewhere herein. The conjunctiva of the eye is dissected to expose the sclera. The first portion 14 of tube 12 is then inserted into the vitreous cavity through a small opening in the sclera of the eye. The stabilizer element 36 is sutured to the sclera and an adhesive is placed at the interface between the sclera and the stabilizer element to secure the stabilizer element in place. The conjunctiva of the eye is then closed over the tube 12 and stabilizer element 36.

Turning now to FIG. 6, the position of the apparatus 10 during intraocular pressure monitoring is shown. The catheter 22 extends outside the eye orbit and beneath the skin of the animal back to the transducer 30.

The rigid tube 12 is effective in maintaining a constant or consistent angular orientation of the catheter 22 in the eye. The stabilizer member 36 is effective to assist in maintaining a constant or consistent angular orientation of the catheter 22 and tube 12 in the eye and/or in maintaining the position of the catheter 22 and tube 12 in the eye. Together, the rigid tube 12 and stabilizer element 36 are effective in maintaining the angular orientation and position of the catheter 22 and tube 12 in the eye, which results in substantial advantages, for example, reduced stress/trauma on the animal, more consistent IOP data monitoring, advantageously longer periods of time during which IOP measurements can be obtained and the like.

The structure of the rigid tube 12 and/or stabilizer element 36 reduces, or even substantially prevents, undesirable motions or displacements of the distal end of catheter 22 in the eye, for example, motions or displacements resulting from blinking or other normal movements of the eye. Because the catheter is more effectively maintained in its original angular orientation and position for an extended period of time, for example, up to about six months or about one year or longer, the measurements of IOP obtained are more accurate or consistent, and therefore more useful, relative to measurements obtained with a similar apparatus without the rigid tube and/or stabilizer element, that is an apparatus the flexible catheter of which is prone to change position in the eye over time.

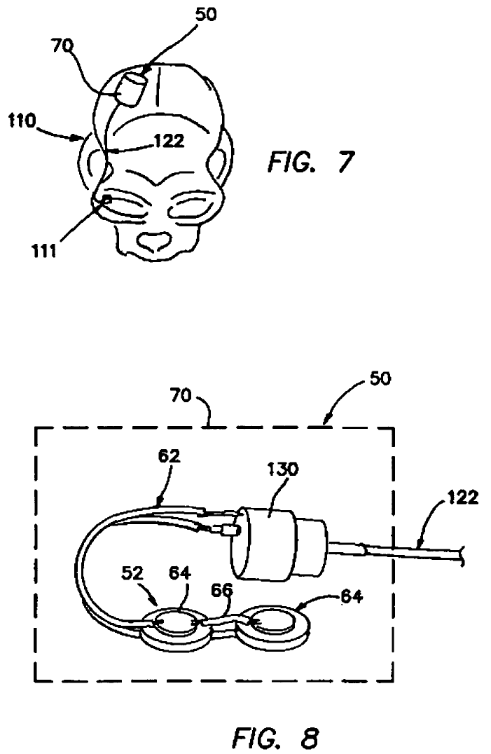

Turning now to FIG. 7, another apparatus 110 in accordance with the invention is shown. Apparatus 110 is substantially the same as apparatus 10, with the primary difference being that rather than including the conventional pressure sensor assembly 28 shown in FIG. 1, an alternative pressure sensor assembly 50 is provided. Except as expressly described herein, apparatus 110 is similar to apparatus 10 and features of apparatus 110 which correspond to features of apparatus 10 are designated by the corresponding reference numerals increased by 100.

The transducer catheter 122 of pressure sensor assembly 50 is connected to the fixation assembly 111 and has substantially the same function as catheter 22 in conventional pressure sensor assembly 28. The pressure sensor assembly 50 (shown in greater detail in FIG. 8) includes a power source 52 coupled to transducer 130 by connectors 62. Transducer 130 is connected to catheter 122.

Advantageously, pressure sensor assembly 50 includes a biocompatible housing 70 which contains the transducer 130 and power source 52 and all remaining components of a pressure sensor used to sense intraocular pressure at the distal end of catheter 122.

Preferably, power source 52 comprises a plurality of batteries 64, for example two batteries 64 connected in parallel by connectors 66. This feature of the invention advantageously provides for longer operation of the apparatus 110 relative to a substantially identically structured apparatus having only a single battery, for example, conventional pressure sensor assembly 28.

As shown, the batteries 64, transducer 130 and connectors 66, are all enclosed within biocompatible housing 70. Advantageously, the entire apparatus 110 is sized and structured to be placed, for example implanted, substantially completely in a body of a human or animal, such as shown in FIG. 7.

Turning now to FIG. 9, alternate apparatus 210 in accordance with the invention is shown. Except as expressly described herein, apparatus 210 is similar to apparatus 10 and features of apparatus 210 which correspond to features of apparatus 10 are designated by the corresponding reference numerals increased by 200.

Apparatus 210 is structured and functions substantially the same as apparatus 10, with the primary difference being that rather than comprising silicone polymeric stabilizer member 36 shown in FIG. 1, an alternative stabilizer member 80 is provided. Stabilizer member 80 comprises a planar member, for example a disc 81 made of a biocompatible material such as a biocompatible metal or biocompatible metal based material. Disk 81 may include one or more apertures, notches or other structure (not shown) for receiving first portion 214 of tube 212 during assembly of apparatus 210.

In one specific embodiment of apparatus 210, first tube portion 214 has a length of about 3 mm, second tube portion 216 has a length of about 3 mm, and disk 81 is substantially circular and has a diameter of about 4 mm and a thickness of about 0.5 mm. Distal end 224 of tube 212 is configured for facilitating placement in an eye. For example, distal end 224 has a non-coring tip including a bevel of about 45°.

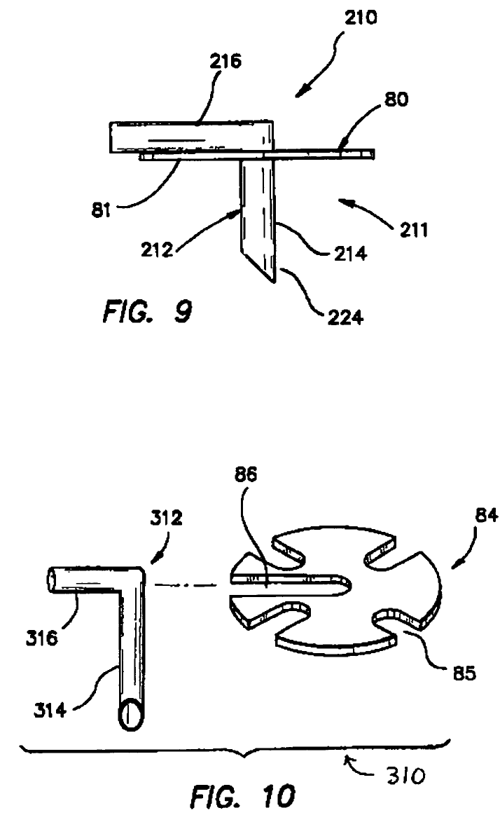

FIG. 10 illustrates another apparatus 310 for use in sensing intraocular pressure in accordance with the invention. Except as expressly described herein, apparatus 310 is similar to apparatus 10 and features of apparatus 310 which correspond to features of apparatus 10 are designated by the corresponding reference numerals increased by 300.

Apparatus 310 is structured and functions substantially the same as apparatus 10, with the primary difference being that rather than tube 312 being derived from a modified G19 needle, tube 312 comprises borosilicate glass.

In addition, apparatus 310 includes a borosilicate glass stabilizer member 84 which includes structure for facilitating fixing or securing the apparatus 312 to the eye. For example, substantially circular stabilizer member 84 includes a plurality of notches 85, for example, radially disposed notches, which provide anchoring points for surgical sutures. In addition, stabilizer member 84 includes a channel 86 extending from a periphery of the stabilizer member 84 to about a center thereof which is sized and structured to secure tube 312 with respect to the stabilizer member 84.

In a specific embodiment, tube 312 includes first portion 314 having a length of about 3 mm, and a second portion 316 having a length of about 1.5 mm. Stabilizer member 84 has a diameter of about 3 mm and a thickness of about 0.5 mm. Notches 85 are about 0.75 mm to about 1 mm in length.

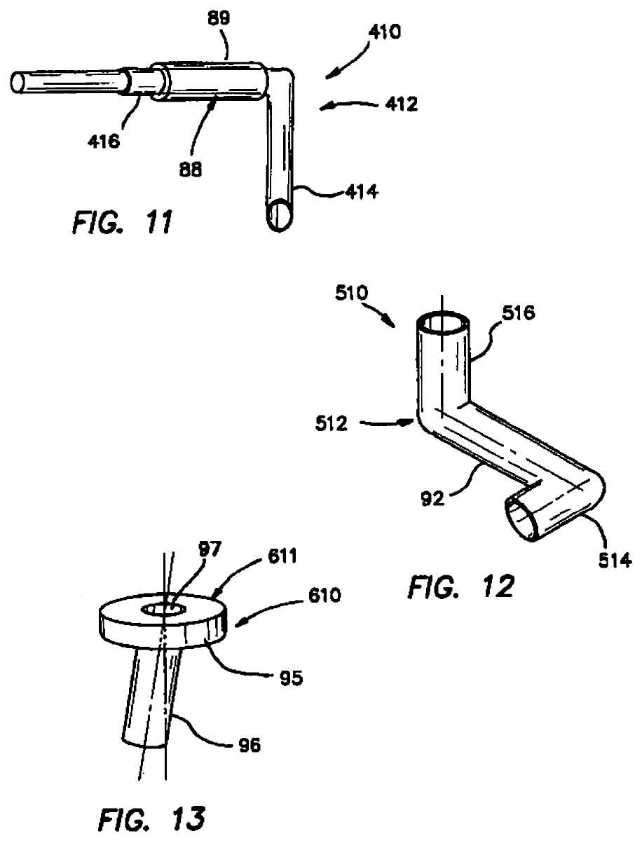

Turning now to FIG. 11, another apparatus 410 in accordance with the invention is shown. Except as expressly described herein, apparatus 410 is similar to apparatus 10 and features of apparatus 410 which correspond to features of apparatus 10 are designated by the corresponding reference numerals increased by 400.

Apparatus 410 is structured and functions substantially the same as apparatus 10, with the primary difference being the stabilizer member 36 is replaced by alternative stabilizer member 88. In this embodiment, stabilizer member 88 comprises a sheath 89 which conforms with, is fitted to and circumscribes at least a portion or substantially all of second portion 416 of tube 412. Sheath 89 is structured to provide a secure site for anchoring sutures to the eye, for example, the sclera of the eye. Preferably, sheath 89 comprises a biocompatible material, for example a silicone material, another biocompatible polymeric material or other suitable material.

In a specific embodiment, tube 412 comprises a modified gold plated G19 needle with first portion 414 being about 3 mm in length an second portion 416 being about 3 mm in length. Sheath 89 has an outer diameter of about 1.7 mm and an inner diameter of about 0.7 mm.

FIG. 12 shows an additional embodiment of the invention. Except as expressly described herein, apparatus 510 is similar to apparatus 10 and features of apparatus 510 which correspond to features of apparatus 10 are designated by the corresponding reference numerals increased by 500.

Apparatus 510 is structured and functions substantially the same as apparatus 10, with the primary difference being that apparatus 510 does not include an enlarged stabilizer member 36 but rather is stabilized in the eye by a configuration and structure of the tube 512 itself. Apparatus 510 generally comprises a tube 512 including a first portion 514 structured to be placed in a sclera and a second 516 portion structured to receive a catheter through a proximal opening thereof. Tube 512 further includes a third portion 92 located between the first portion 514 and second portion 516 and disposed at a first angle to first portion 514 and a second angle to second portion 516. All of first portion 514, second portion 516 and third portion 92 define a hollow through space sized to allow a flexible catheter of a pressure sensor to pass in or in fluid communication with the hollow through space.

In a specific embodiment of the invention, first portion 514 and third portion 92 are disposed at an angle of about 90°, and second portion 516 and third portion 92 are disposed at an angle of about 90°. In other words, tube 512 lies along 3 different geometrical axes (X-axis, Y-axis and Z-axis). Although third tube portion 92 can be considered a stabilizer in accordance with the present invention, this geometrical configuration allows for stability of apparatus 510 in the eye without the addition of an enlarged stabilizer member. Tube 512 may be entirely derived from a single, gold electroplated G19 needle.

Another distinction between apparatus 510 and apparatus 10 is that apparatus 510 includes a relatively blunt or non-beveled distal end 93 as shown. Alternatively, the distal end may be beveled.

Referring now to FIG. 13, still another embodiment of the invention is shown. Except as expressly described herein, apparatus 610 is similar to apparatus 10 and features of apparatus 610 which correspond to features of apparatus 10 are designated by the corresponding reference numerals increased by 600.

Apparatus 610 is structured and functions substantially the same as apparatus 10, with the primary difference being that apparatus 610 does not include a tube having angularly disposed first and second portions. Apparatus 610 comprises an enlarged stabilizer portion 95 and a tube portion 96 depending therefrom, the stabilizer portion 95 and the tube portion 96 defining a hollow through space 97 sized and structured to allow a flexible catheter of a pressure sensor to pass in or in fluid communication with the hollow through space 97.

In a specific embodiment, tube portion 96 is derived from an electroplated gold G19 needle. Stabilizer portion 95 comprises any suitable biocompatible material, for example, as described elsewhere herein. Suitable adhesive may be provided for securing stabilizer portion 95 and tube portion 96. Tube portion 96 is about 3 mm in length and stabilizer portion 95 is about 3 mm in diameter. In a preferred embodiment for use in measuring intraocular pressure, tube portion 96 depends from stabilizer portion 95 at a fixed angle of about 70°. This relative positioning of tube portion 96 with respect to stabilizer portion 95 effectively directs distal end 624 of tube portion 96 away from a lens of an eye when the apparatus 610 is located in the eye.

The present invention also provides methods for sensing intraocular pressure. Such methods comprise placing a flexible catheter of a pressure sensor used to sense intraocular pressure in or in fluid communication with a hollow through space defined by a rigid tube, such as shown and described elsewhere herein, including a first portion and a second portion positioned at a fixed angle relative to the first portion. The distal end portion of the rigid tube is introduced or inserted or placed into an eye of a human or animal. The pressure sensor is employed to sense the intraocular pressure in the eye of the human or animal, for example on a continuous and/or long term basis, for example, up to about six months or about 1 year or longer.

The following, non-limiting, Examples illustrate certain aspects of the present invention.

### EXAMPLE 1

A G19 regular wall needle, having a beveled distal end, is heated and then bent to form a bent needle having a distal or first portion about 4 mm in height and a proximal or second portion such that the two portions are oriented at an angle of 90° relative to each other. The bent needle is tested to ensure that the hollow through space formed by the needle remains open, that is has not been substantially or even totally occluded by the bending.

A stabilizer element, made of silastic polymeric material and in the form of a disc as shown in FIGS. 1 to 4, is provided. The beveled distal tip of the bent needle is passed into and through the stabilizer element to form an assembly as shown in FIG. 3. The bent needle, having an open hollow through space and the stabilizer element, is then subjected to a cutting operation in which the proximal or second portion is cut to a length of about 4 mm. This assembly is then subjected to autoclaving to produce a sterilized rigid tube/stabilizer assembly ready for use in the surgical procedure described hereinafter.

Albino rabbits (2-4 kg) are sedated with ketamine, intubated, and anesthetized with isoflurane. The scalp is prepared for aseptic surgery, followed by a 4 cm incision and tissue blunt-dissection to form a pocket in the underlying fascia to contain a pressure sensor assembly, such as pressure transducer 30 shown in FIG. 1. The catheter 122 is then guided subcutaneously, for example, using a gauge 13, 5.5 inch long needle, to the orbit of the eye.

The distal end of the catheter is then passed into the proximal end of the bent needle and extends into the through hollow space of the bent needle up to about the bend in the needle. A drop of biocompatible adhesive, for example, cyanoacrylate adhesive, is placed on the junction between the catheter and the bent needle to secure and pressure seal the catheter to the bent needle. The distal portion of the through hollow space of the bent needle is filled with a conventional biocompatible gel so that the pressure at the distal tip of the bent needle will be transmitted to the distal end of the catheter to facilitate accurate IOP sensing.

The conjunctiva of the eye is then dissected to expose the sclera. A mark is made about 4 mm from the limbus. A hole or puncture in the sclera is made at this mark, for example, using a conventional MVR blade. The distal portion of the bent needle is then placed in the vitreous of the eye through the hole or puncture. The stabilizer element is sutured to the sclera and a conventional skin adhesive is placed on the stabilizer element at the interface between the sclera and the stabilizer element. The conjunctiva is then closed over the bent needle and stabilizer element.

After surgery, receivers are placed into the animal cages and connected to a computer running software, for example, DSI Dataquest software, for data IOP capture. Animals are housed in a 12 hours light, 12 hours dark cycle: 6:00 AM ON, 6:00 PM OFF. After a recovery and stabilization period of 2-4 weeks, timolol was topically applied to the instrumented eye of the rabbits.

Results: IOP in rabbits is lower during the day than at night. IOPs are lower in the dark phase than the light phase by a maximum of 8±2.3 mm Hg and occur 3 hours following lights-out. Timolol (0.5%) applied at 9:00 am in the morning lowers IOP 35% during the day only. There is no drug effect at night. Upon dissection of the eye following the experiment, it is discovered that the catheter distal end had not migrated or changed position to any significant degree.

### EXAMPLE 2

Example 1 is repeated except that the transducer assembly including two batteries in parallel, as shown in FIG. 8, is employed. Substantially similar results are obtained.

The IOP of the animals is monitored for a period of eight months with no need to replace the power supply in the transducer assembly.

While this invention has been described with respect to various specific examples and embodiments, it is to be understood that the invention is not limited thereto and that it can be variously practiced within the scope of the following claims.

What is claimed is:

1. An apparatus for use in sensing intraocular pressure, the apparatus comprising:
a rigid tube including a first portion and a second portion positioned at a fixed angle relative to the first portion, the tube defining a hollow through space sized and structured to allow a flexible catheter of a pressure sensor used to sense intraocular pressure to pass in or in fluid communication with the hollow through space.

2. The apparatus of claim 1 which is structured to be placed substantially completely in a body of a human or animal.

3. The apparatus of claim 1 wherein the first and second portions define an angle therebetween in a range of about 30° to about 150°.

4. The apparatus of claim 1 wherein the first and second portions define an angle therebetween in a range of about 65° to about 105°.

5. The apparatus of claim 1 wherein the first and second portions define an angle therebetween of about 90°.

6. The apparatus of claim 1 wherein the first and second portions define an angle therebetween of about 70°.

7. The apparatus of claim 1 wherein the tube further includes a third portion positioned at a fixed angle relative to the first portion.

8. The apparatus of claim 1 wherein the tube further includes a third portion between the first portion and second portion, the third portion being positioned at a fixed angle relative to the first portion and positioned at a fixed angle relative to the second portion.

9. The apparatus of claim 1 which further comprises a stabilizer secured to the tube, the stabilizer being configured to be effective in maintaining a desired orientation of the tube in an eye into which the tube is inserted.

10. The apparatus of claim 9 wherein the stabilizer is structured to provide a site for anchoring sutures.

11. The apparatus of claim 9 wherein the stabilizer comprises a biocompatible polymeric material.

12. The apparatus of claim 9 wherein the stabilizer comprises a silicone material.

13. The apparatus of claim 9 wherein the stabilizer substantially surrounds a region of the tube at which the first and second portions meet.

14. The apparatus of claim 9 wherein the stabilizer comprises a sheath disposed around a portion of the tube.

15. The apparatus of claim 1 which further comprises a flexible catheter of a pressure sensor used to sense intraocular pressure, the catheter being biocompatible and sized and structured to pass in or in fluid communication with the hollow through space.

16. The apparatus of claim 15 which includes a battery assembly structured and positioned to power the pressure assembly.

17. The apparatus of claim 16 wherein the battery assembly comprises two or more batteries in parallel.

18. The apparatus of claim 15, which is structured to be placed substantially completely in a body of a human or animal.

19. A method for measuring intraocular pressure, the method comprising:
placing a flexible catheter of a pressure sensor used to sense intraocular pressure in or in fluid communication with a hollow through space defined by a rigid tube including a first portion and a second portion positioned at a fixed angle relative to the first portion;
introducing a distal end portion of the rigid tube into an eye of a human or animal; and
employing the pressure sensor to sense the intraocular pressure in the eye of the human or animal.

20. The method of claim 19 wherein the first and second portions define an angle therebetween in a range of about 30° to about 150°.

21. The method of claim 19 wherein the first and second portions define an angle therebetween of about 90°.

22. The method of claim 19 wherein the tube further includes a third portion positioned at a fixed angle relative to the first portion.

23. The method of claim 19 wherein a stabilizer is secured to the tube and is effective in maintaining a desired orientation of the tube in the eye after the introducing step.

24. The method of claim 19 wherein the employing step comprises powering the pressure sensor by a battery assembly.

25. An apparatus for use in sensing intraocular pressure, the apparatus comprising:
a stabilizer portion and a tube portion depending from the stabilizer portion, the tube portion defining a hollow through space sized and structured to allow a flexible catheter of a pressure sensor used to sense intraocular pressure to pass in or in fluid communication with the hollow through space.

26. The apparatus of claim 25 wherein the stabilizer portion and the tube portion define an angle therebetween in a range of about 65° to about 105°.

27. The apparatus of claim 25 wherein the stabilizer portion and the tube portion define an angle therebetween of about 70°.

28. The apparatus of claim 25 wherein the stabilizer portion is structured to provide a site for anchoring sutures.

29. The apparatus of claim 25 wherein the stabilizer portion includes at least one notch that provides a site for anchoring sutures.

30. The apparatus of claim 25 which further comprises a flexible catheter of a pressure sensor used to sense intraocular pressure, the catheter being biocompatible and sized and structured to pass in or in fluid communication with the hollow through space.
